(19) United States
(12) Plant Patent Application Publication
(10) Pub. No.: US 2003/0024019 P1
(43) Pub. Date: Jan. 30, 2003

(54) FLORIBUNDA ROSE PLANT NAMED 'POULDAHLE'

(75) Inventors: L. Pernille Olesen, Fredensborg (DK); Mogens N. Olesen, Fredensborg (DK)

Correspondence Address: Mogens N. Olesen, Poulsen Roser Pacific, Inc., 2550 Old Military Road, Central Point, OR, 97502, US

(73) Assignee: Poulsen Roser ApS.

(21) Appl. No.: 09/774,822

(22) Filed: Jan. 30, 2001

(51) Int. Cl. A01H 5/00

(52) U.S. Cl. PLT/148

(57) ABSTRACT

A new floribunda garden rose plant which has abundant, light pink to peach flowers and attractive foliage. This new and distinct variety has shown to be uniform and stable in the resulting generations from asexual propagation.

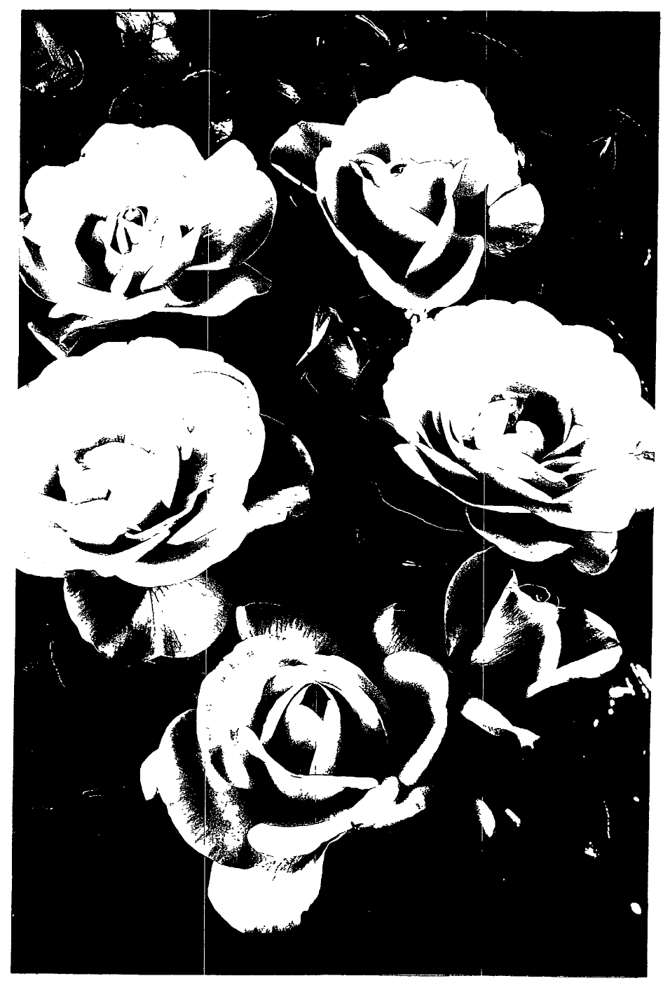

### SUMMARY OF THE INVENTION

The present invention constitutes a new and distinct variety of garden rose plant which originated from a controlled crossing between ‘St. Helena’ (CANlish) and an ‘unnamed seedling’, both unpatented varieties. The two parents were crossed during the summer of 1993 and the resulting seeds were planted in a controlled environment in Fredensborg, Denmark. The new variety is named ‘POULdahle’.

The new rose may be distinguished from its seed parent, ‘St. Helena’, by the following combination of characteristics:

1. ‘POULdahle’ is more free-flowering than the seed variety.

2. ‘POULdahle’ has soft pink to peach flowers, while ‘St. Helena’ has lilac pink flowers.

3. ‘POULdahle’ exhibits thicker and more glossy foliage than ‘St. Helena’.

The new variety may be distinguished from its pollen parent, ‘an unnamed seedling’ by the following combination of characteristics:

1. ‘POULdahle’ contains more flowers per plant when compared to the pollen parent.

2. ‘POULdahle’ exhibits thicker and more glossy foliage than the pollen parent.

The objective of the hybridization of this rose variety was to create a new and distinct variety for garden use with unique qualities, such as:

1. Uniform and abundant flowers;

2. Vigorous, but compact growth when propagated both as a budded rose and on its own roots;

3. Disease resistance.

This combination of qualities is not present in previously available commercial cultivars of this type and distinguish ‘POULdahle’ from all other varieties of which we are aware.

As part of their rose development program, L. Pernille Olesen and Mogens N. Olesen germinated the seeds from the aforementioned hybridization during winter 1993 and conducted evaluations on the resulting seedlings in a controlled environment in Fredensborg, Denmark.

‘POULdahle’ was selected in the spring 1994 by the inventors as a single plant from the progeny of the aforementioned hybridization.

Asexual reproduction of ‘POULdahle’ by traditional budding and rooted cuttings was first done by L. Pernille and Mogens N. Olesen in their nursery in Fredensborg, Denmark in August, 1994. This initial and other subsequent asexual propagations conducted in controlled environments have demonstrated that the characteristics of ‘POULdahle’ are true to type and are transmitted from one generation to the next.

### BRIEF DESCRIPTION OF THE DRAWING

The accompanying color illustration shows as true as is reasonably possible to obtain in color photographs of this type ‘POULdahle’, potted rose plant. Specifically illustrated in SHEET 1 are ‘POULdahle'’ foliage, flower buds, partially opened buds, and open blooms.

### DETAILED DESCRIPTION OF THE VARIETY

The following is a description of ‘POULdahle’, as observed in its growth during trials at the testing station Prufstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULskov’, a rose variety from the same inventors described and illustrated in U.S. Plant Pat. No. 9,062 and issued on Feb. 28, 1995 are compared to ‘POULdahle’ in Chart 1.

CHART 1

| | ‘POULdahle’ | ‘POULskov’ |
|---|---|---|
| Color of open | Red Group 56D | Red Group 49D-56B |
| flower, | | |
| innermost | | |
| petals, | | |
| middle zone. | | |
| Petalage | Semi-double | Very double |
| Upon opening, | Yellow Group 8C | Yellow-Green |
| color basal | | Group 154D to |
| petal spot, | | Yellow Group 4D |
| outer side | | |

Parents:

Seed parent.—‘St. Helena’.

Pollen parent.—‘Unnamed seedling’.

Classification:

Botanical.—Rosa hybrida.

Commercial.—Floribunda.

### FLOWER AND FLOWER BUD

Blooming habit: Continuous.

Flower bud:

Bud form.—Ovate.

Color.—As sepals unfold, Orange Group 29D.

Sepals.—Weak appendages.

Receptacle.—Size: Small to medium.

Peduncle.—Surface: Average to above average abundance of hairs and prickles.

Borne.—Singularly. Very few to few flowering shoots.

Flower bloom:

Fragrance.—Weak.

Size.—Average flower diameter is 100-120 mm when open.

Form.—Shape of flower when viewed from the side: Upper part: Flat. Lower part: Flat. Viewed from above: Irregularly rounded.

Petalage.—Semi-double. Average range: 18-20 petals under normal conditions.

Color:

Petals.—Inner Side: Middle zone: Red Group 56D. Marginal zone: Red Group 56D. Basal petal spot: Yellow Group 8B-C. Outer side: Middle zone: Red Group 56D. Marginal zone: Red Group 56D. Basal petal spot: Yellow Group 8C.

General Tonality: On open flower Red Group 36B to Orange Group 29D. No change in the general tonality at the end of the 5th day.

Petals:

Petal reflex.—Somewhat reflexed.

Undulation of margin.—Weak to medium.

Size.—Medium to large.

Quantity.—Very few to few.

Thickness.—Average.

Reproductive Organs:

Filaments.—Color: Yellow.

Hips.—Shape: Pitcher-shaped.

### PLANT

Plant growth: Moderate, upright to bushy. When grown as a budded field grown plant on Rosa multiflora understock, the average height of the plant is 60-80 cm and the average width is 60 cm.

Stems:

Thorns.—Incidence: Moderate. Size: Small. Shape: Concave.

Plant foliage:

Leaf size.—Small to medium.

Glossy.—Upper side medium glossiness.

Color.—Upper Leaf Surface: Dark green. Juvenile foliage: Exhibits anthocyanin on young shoots. Anthocyanin intonation: Color: Reddish brown.

Plant leaflets:

Leaflet.—Cross Section: Flat. Margin Undulation: very weak to weak. Other: Surface glossy.

Terminal Leaflet.—Length of blade: Medium. Width of blade: Narrow to medium. Shape: Rounded.

Disease resistance: Above average resistance to mildew, rust, black spot, and Botrytis under normal growing conditions in Fredensborg, Denmark and Hannover, Germany.

Cold Hardiness: The variety ‘POULdahle’ has been found to be cold hardy in Fredensborg, Denmark and Hannover, Germany.

1. A new and distinct variety of rose plant of the floribunda rose class, substantially as herein illustrated and described as a distinct and novel rose variety due to its abundant light pink to peach flowers, disease resistance, and extended period of bloom.
